(19) United States
(12) Patent Application Publication
(10) Pub. No.: US 2021/0301314 A1
(43) Pub. Date: Sep. 30, 2021

(54) PREPARATION METHOD AND APPLICATION OF SEA CUCUMBER PROTEIN PEPTIDES

(71) Applicants: Chenland Nutritionals Inc., Pomona, CA (US); Qingdao Chenland Pharmaceutical Technology Development Co., Ltd., Qingdao (CN)

(72) Inventors: Shengcan Zou, Qingdao (CN); Changwei Wang, Qingdao (CN); Yanmei Wang, Qingdao (CN); Shanglong Wang, Qingdao (CN); Tingfeng Cao, Qingdao (CN); Bafang Li, Qingdao (CN)

(73) Assignees: Chenland Nutritionals Inc., Pomona, CA (US); Qingdao Chenland Pharmaceutical Technology Development Co., Ltd., Qingdao (CN)

(21) Appl. No.: 17/180,727

(22) Filed: Feb. 20, 2021

Related U.S. Application Data
(63) Continuation of application No. PCT/CN2020/088987, filed on May 7, 2020.

(30) Foreign Application Priority Data
Mar. 26, 2020 (CN) 2020102247803

(51) Int. Cl. C12P 21/06; C07K 1/34; A23J 3/34; A61K 8/64; A23L 33/18

(52) U.S. Cl. CPC C12P 21/06; C07K 1/34; A23L 33/18; A61K 8/64; A23J 3/341

(57) ABSTRACT

A preparation method and an application of sea cucumber protein peptides are disclosed. The disclosure develops a preparation method of sea cucumber protein peptides with simple operation and excellent product quality by optimizing raw materials and enzymolysis extraction process of sea cucumber protein peptides. On the one hand, the problem that the deodorization and decolorization effect of the sea cucumber peptide hydrolysate of the prior art is not obvious is solved. On the other hand, the obtained sea cucumber protein peptides can be processed into nutritional supplements and used as raw materials for preparing health care products or cosmetics for enhancing immunity, relieving physical fatigue, resisting oxidation and aging, which has good development and utilization potential.

### TECHNICAL FIELD

The present disclosure belongs to the field of intensive processing of aquatic products, relates to preparation of protein peptides, and more specifically, to a simple preparation method of a series of sea cucumber protein peptides, and discusses the antioxidant activity of sea cucumber protein peptides and its application in food and daily cosmetics.

### BACKGROUND

Sea cucumbers are rich in nutrients and have been a noble food and a remedy for the prevention of diseases since ancient times in China. Sea cucumbers have the functions of delaying aging of human body, lowering blood pressure, resisting tumor, preventing cardiovascular and cerebrovascular diseases, improving immunity and beautifying skin due to its nutrient substances such as protein polypeptide, sea cucumber polysaccharide and sea cucumber saponin. Therefore, through scientific means to fully retain the most valuable active ingredients and various nutrients in sea cucumbers, they can be easily absorbed by people, which is more conducive to the full play of the value of sea cucumbers.

With the development of deep processing technology, sea cucumbers are deeply processed into different molecular weight peptide products to meet the different needs of consumers. However, at present, most of the processing technology of sea cucumbers needs to adjust the optimal pH value repeatedly, which is not only complicated to operate and easy to corrode the equipment, but also brings a large number of inorganic ions into the products, ultimately affecting the product food taste and quality. And the solution of most peptide products at present is dark and fishy, which greatly affects the sales of peptide products.

Therefore, how to provide a preparation method of sea cucumber protein peptides with simple operation and excellent quality is an urgent technical problem for those skilled in the art.

### SUMMARY

In view of the above, an object of the present disclosure is to provide a preparation method of sea cucumber protein peptides according to the problems in the prior art. The sea cucumber protein peptides prepared by the method have the advantages of controllable molecular weight, light color, no fishy smell and high antioxidant activity.

Technical solutions of the present disclosure are specifically described as follows.

A preparation method of sea cucumber protein peptides, including:
(1) pretreatment of raw material: eviscerating, washing, drying and grinding fresh Cucumana frondos into a homogenate;
(2) enzymolysis: adding water to the homogenate of step (1) and determining pH, and then adding a protease for enzymolysis to obtain a sea cucumber peptide hydrolysate;
(3) purification and drying: subjecting the sea cucumber peptide hydrolysate of step (2) to boiling water bath to inactivate enzyme, followed by filtering, decolorizing and spray drying to obtain the sea cucumber protein peptides.

Cucumaria frondos are used as the raw materials of the sea cucumber protein peptide, which are wild sea cucumbers grown in the deep-sea waters of Iceland close to the arctic circle. And the growth environment of the Cucumaria frondos is special, complex, pollution-free and pure wild. The Cucumaria frondos are rich in nutrition, and their protein contents are more than 75% of the dry matters.

In addition, four different kinds of sea cucumber raw materials are subjected to quality analysis to determine the source of the raw materials for preparing the sea cucumber protein peptides, and the specific experimental operation is as follows. Finally, fresh Cucumaria frondos were selected as the raw materials for the sea cucumber protein peptides based on comprehensive analysis of nutritional composition, cost, and specificity of source environment.

Preferably, the protease in step (2) is a flavourzyme, a papain, a pancreatin, a neutral protease or an alkaline protease, and an addition of the protease is 500˜6000 U/g.

Further, in step (2), an enzymolysis temperature is 40° C.˜60° C., and an enzymolysis time is 0.5˜6 h.

Further, a mass ratio of the homogenate to the water is 1:(2˜2.5), and the pH of the enzymolysis is 6˜8.

Preferably, in step (3), a temperature of the boiling water bath is 95° C.˜100° C., and a time of enzyme inactivation is 10˜15 min.

In addition, a specific operation of the purification is as follows: passing the hydrolysate through an ordinary filter paper, an 1.2 μm filter membrane and an 0.8 μm filtering membrane in turn, adding a decolourant into the filtered hydrolysate for decolorization, centrifuging the decolorized hydrolysate under hot, passing the centrifuged hydrolysate through an 0.8 μm filtering membrane, and then filtering with an celite filter cake, after passing through a 0.45 μm filter membrane, ultrafiltering with a 10 k ultrafiltration membrane to obtain a purification filtrate.

Preferably, the decolourant is activated carbon or diatomite, and an addition of the decolourant is 1˜3% of the volume of the hydrolysate, and a decolorizing temperature is 60° C.˜80° C., and a decolorizing time is 0.5˜1.5 h.

The present disclosure adopts directional enzymolysis technique to obtain Cucumaria frondos protein peptides with different molecular weights. Studies have shown that the strength of antioxidant activity of protein peptides from Cucumaria frondos is related to the molecular weight of protein peptides, and the rule is showed that the smaller the molecular weight, the stronger the antioxidant activity. The peptides with molecular weight segments below 1000 Da are called small active peptides, also called small peptides or oligopeptides; the peptides with molecular weight segments 1000 Da-10000 Da are called polypeptides, and polypeptides with more than 50 amino acids are called protein (>10000 Da).

Proteins, polypeptides and oligopeptides play a key role in the process of human growth and development, metabolism, diseases as well as aging and death. Exogenous intake of polypeptides and oligopeptides has many benefits on human physiological functions, such as increasing energy, enhancing immunity, protecting heart and brain vessels, and delaying aging. At present, the function of anti-aging by reducing oxygen radicals (O²⁻.) and hydroxyl radicals (.OH) has attracted much attention from researchers.

The present disclosure also claims to protect an application of the sea cucumber protein peptides prepared by the preparation method mentioned above in food and daily cosmetics.

Compared with the prior art, the disclosure provides a preparation method and application of sea cucumber protein peptides with the following beneficial effects.

The disclosure develops a preparation method of sea cucumber protein peptides with simple operation and excellent product quality by optimizing raw materials and enzymolysis extraction process of sea cucumber protein peptides. On the one hand, the problem that the deodorization and decolorization effect of the sea cucumber peptide hydrolysate of the prior art is not obvious is solved. On the other hand, the obtained sea cucumber protein peptides can be processed into nutritional supplements and used as raw materials for preparing health care products or cosmetics for enhancing immunity, relieving physical fatigue, resisting oxidation and aging, which has good development and utilization potential.

### BRIEF DESCRIPTION OF THE DRAWINGS

In order to explain the embodiments of the present disclosure or the technical solutions in the prior art more clearly, the following drawings that need to be used in the description of the embodiments or the prior art will be briefly introduced. Obviously, the drawings in the following description are only embodiments of the present disclosure. For those of ordinary skill in the art, other drawings can be obtained based on the drawings disclosed without creative work.

FIG. 1 is a flow chart for the disclosed preparation method of sea cucumber protein peptides.

FIG. 2 shows the curve of the content of amino free nitrogen after enzymolysis by different proteases.

FIG. 3 shows the effect of pH and temperature on the degree of hydrolysis.

FIG. 4 shows the effect of pH and enzyme dosage on the degree of hydrolysis FIG. 5 shows the effect of temperature and enzyme dosage on the degree of hydrolysis.

### DETAILED DESCRIPTION OF EMBODIMENTS

Technical solutions of the present disclosure will be clearly and completely described below with the drawings of the embodiments. Obviously, the described embodiments are only part of the embodiments of the present invention, not all of them. Other embodiments made by those skilled in the art without sparing any creative effort should fall within the scope of the disclosure.

The embodiments disclose a preparation method of sea cucumber protein peptides with simple operation and excellent quality.

The disclosure will be further described below with reference to the embodiments. It should be understood that these embodiments are merely illustrative of the disclosure, and are not intended to limit the disclosure. Any improvement and modification made by those skilled in the art without departing from the spirit of the disclosure should still fall within the scope of the disclosure.

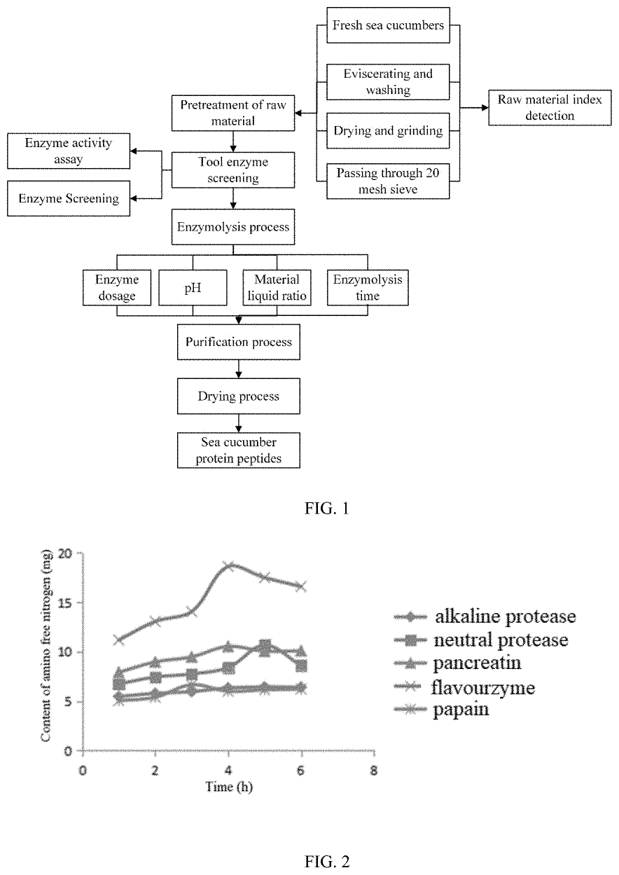

As shown in FIG. 1, the disclosure disclosed a preparation method of sea cucumber protein peptides, including:
(1) pretreatment of raw material: eviscerating, washing, drying and grinding fresh Cucumana frondos into a homogenate;
(2) enzymolysis: adding water to the homogenate of step (1) and determining pH, and then adding a protease for enzymolysis to obtain a sea cucumber peptide hydrolysate;
(3) purification and drying: subjecting the sea cucumber peptide hydrolysate of step (2) to boiling water bath to inactivate enzyme, followed by filtering, decolorizing and spray drying to obtain sea cucumber protein peptides.

Further, the protease in step (2) is a flavourzyme, a papain, a pancreatin, a neutral protease or an alkaline protease, and an addition of the protease is 500˜6000 U/g. An enzymolysis temperature is 40° C.˜60° C., and an enzymolysis time is 0.5˜6 h. And a mass ratio of the homogenate to the water is 1:(2˜2.5), and the pH of the enzymolysis is 6˜8.

Further, in step (3), a temperature of the boiling water bath is 95° C.˜100° C., and a time of enzyme inactivation is 10˜15 min.

In addition, a specific operation of the purification is as follows: passing the hydrolysate through an ordinary filter paper, an 1.2 μm filter membrane and an 0.8 μm filtering membrane in turn, adding a decolourant into the filtered hydrolysate for decolorization, centrifuging the decolorized hydrolysate under hot, passing the centrifuged hydrolysate through an 0.8 μm filtering membrane, and then filtering with an celite filter cake, after passing through a 0.45 μm filter membrane, ultrafiltering with a 10 k ultrafiltration membrane to obtain a purification filtrate.

Further, the decolourant is activated carbon or diatomite, and an addition of the decolourant is 1˜3% of the volume of the hydrolysate, and a decolorizing temperature is 60° C.˜80° C., and a decolorizing time is 0.5˜1.5 h.

According to the above steps, the following single factor experiments were carried out to obtain the best preparation conditions of sea cucumber protein peptides. The specific operation is as follows.

Experiment 1: Protease Screening
(1) Enzyme activity assays were performed for the different proteases. Assay method: determining according to the protease preparation method specified in the national standard GB/T 23527-2009. The specific enzyme activity assay results were shown in Table 1:

TABLE 1
Enzyme activity assay results

| Protease | Enzyme activity (U/g) |
|---|---|
| Alkaline protease | 1.97*10⁵ |
| Neutral protease | 5.01*10⁴ |
| Pancreatin | 1.7*10⁵ |
| Flavourzyme | 1.6*10⁴ |
| Papain | 1.16*10⁵ |

(2) Several kinds of different proteases were selected for enzymolysis of fresh sea cucumbers, and the content of amino free nitrogen after enzymolysis by different proteases was determined, and suitable proteases were screened out.

Where, the enzymolysis conditions of protease screening were as follows: 10 g of sea cucumber homogenate was supplemented with 20 g of double distilled water with 4000 U/g of protease added, pH was adjusted to the optimum pH value of each protease (alkaline: 9, pancreatin: 8, neutral, flavor and papain: 7), 50° C. of enzymolysis lasted for 6 h with sampling on the hour, and determination of the content of amino free nitrogen is performed after enzyme inactivation (95-100° C. for 1015 min), respectively.

The content of amino free nitrogen was determined using the ninhydrin method.

The specific results are shown in FIG. 2 and Table 2.

TABLE 2
The content of amino free nitrogen (mg)

| | 1 h | 2 h | 3 h | 4 h | 5 h | 6 h |
|---|---|---|---|---|---|---|
| Alkaline protease | 5.47 | 5.78 | 5.96 | 6.32 | 6.43 | 6.4 |
| Neutral protease | 6.74 | 7.43 | 7.74 | 8.39 | 10.65 | 8.61 |
| Pancreatin | 7.91 | 8.95 | 9.47 | 10.52 | 10.04 | 10.08 |
| Flavourzyme | 11.17 | 13.04 | 14.04 | 18.6 | 17.48 | 16.57 |
| Papain | 5.04 | 5.39 | 6.65 | 6 | 6.14 | 6.21 |

In conclusion, according to the protease screening results, it was found that the enzymolysis of flavourzyme worked best, so flavourzyme was selected for the next enzymolysis test.

Experiment 2: Optimization of Enzymolysis Extraction Process

In this experiment, the protease was selected as flavourzyme, the enzymolysis time was 3˜5 h. Three factor (pH, temperature, enzyme dosage) tests were designed using the response surface software Design Expert 11. After enzyme inactivation and centrifugation, the hydrolysate was obtained under each factor condition. After the hydrolysate was successively passed through a common filter paper, 1.2 μm filter membrane and 0.8 μm filter membrane, 2% (volume ratio) activated carbon was added. Then the treated hydrolysate was decolorized at 70° C. for 1 h, centrifuged under hot, filtered through 0.8 μm filter membrane, filtered with celite filter cake, filtered through 0.45 μm filter membrane ultrafiltered through 10 k ultrafiltration membrane and spray dried to obtain bright white sea cucumber protein peptides. The test results of the hydrolysis degree, molecular weight and antioxidant evaluation of sea cucumber protein peptides were shown in Table 3.

TABLE 3
The test results of hydrolysis degree, molecular weight and
antioxidant evaluation of sea cucumber protein peptides

| | | | enzyme | hydrolysis | | O²⁻• | •OH |
|---|---|---|---|---|---|---|---|
| | | TEMP | dosage | degree | Mw | clearance | clearance |
| No. | pH | (° C.) | (U) | (%) | (Da) | (%) | (%) |
| 1 | 8 | 60 | 4000 | 24.84 | 868 | 14.97 ± 3.97 | 20.57 ± 0.85 |
| 2 | 6 | 50 | 5000 | 24.33 | 472 | 13.96 ± 7.83 | 24.37 ± 0.38 |
| 3 | 7 | 50 | 4000 | 27.17 | 519 | 12.87 ± 4.30 | 19.95 ± 0.26 |
| 4 | 6 | 40 | 4000 | 25.52 | 608 | 11.86 ± 2.86 | 22.64 ± 2.44 |
| 5 | 6 | 60 | 4000 | 19.6 | 646 | 15.76 ± 1.32 | 25.10 ± 0.36 |
| 6 | 7 | 50 | 4000 | 25.68 | 599 | 14.66 ± 2.86 | 23.96 ± 0.88 |
| 7 | 8 | 50 | 3000 | 22.8 | 431 | 14.04 ± 6.62 | 21.97 ± 1.21 |
| 8 | 7 | 40 | 5000 | 25.78 | 586 | 14.11 ± 4.08 | 23.33 ± 1.03 |
| 9 | 8 | 50 | 5000 | 25.64 | 751 | 17.16 ± 6.40 | 19.68 ± 0.86 |
| 10 | 7 | 50 | 4000 | 29.12 | 761 | 15.99 ± 4.08 | 21.67 ± 0.66 |
| 11 | 7 | 50 | 4000 | 26.58 | 449 | 14.43 ± 0.33 | 25.47 ± 1.16 |
| 12 | 6 | 50 | 3000 | 23.04 | 495 | 18.80 ± 4.52 | 27.13 ± 1.54 |
| 13 | 8 | 40 | 4000 | 23.31 | 547 | 13.96 ± 3.20 | 22.76 ± 2.74 |
| 14 | 7 | 60 | 5000 | 26.28 | 525 | 9.75 ± 2.97 | 24.58 ± 1.51 |
| 15 | 7 | 60 | 3000 | 24.11 | 601 | 8.11 ± 3.97 | 24.26 ± 2.19 |
| 16 | 7 | 50 | 4000 | 28.09 | 477 | 16.30 ± 0.77 | 21.41 ± 1.25 |
| 17 | 7 | 40 | 3000 | 21.19 | 461 | 12.95 ± 2.65 | 27.60 ± 0.55 |

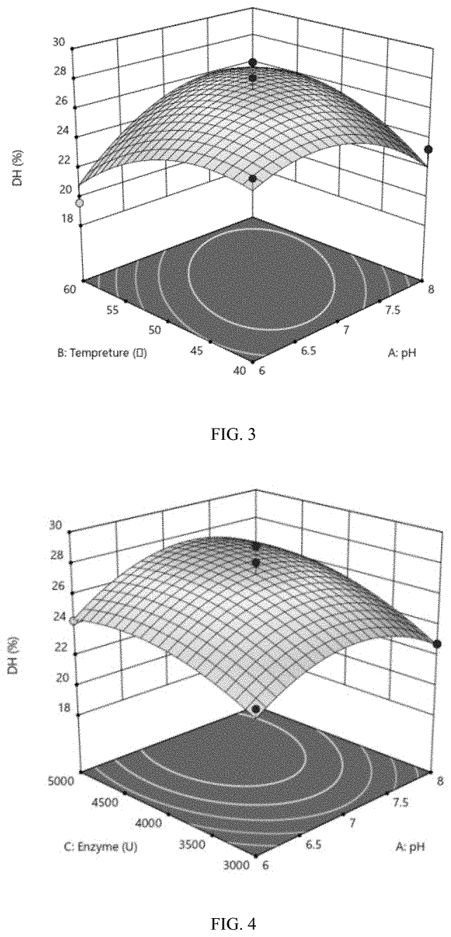

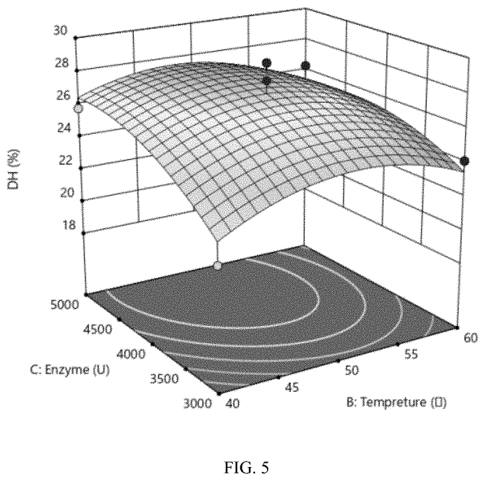

The response experimental results in FIG. 3˜FIG. 5 showed that the optimal preparation process of the sea cucumber protein peptides disclosed by the present disclosure was as follows: the material liquid ratio (the added mass ratio of homogenate to water) was 1:2, the system pH was 7.45, the flavourzyme supplemented was with 4600 U/g protein, the enzymolysis temperature was 49.5° C., and the enzymolysis time was 5 h. The results of antioxidant experiments showed that the strength of the antioxidant activity of Cucumaria frondos protein peptides was related to the molecular weight of protein peptides, and the rule was showed that the smaller the molecular weight, the stronger the antioxidant activity.

The technical schemes of the present disclosure will be further illustrated below in combination with specific embodiments.

It should be noted that the present disclosure is not limited to the following embodiments.

### Embodiment 1

Cucumaria frondos were eviscerated, washed, air dried and homogenized. 10 g of sea cucumber homogenate was weighed, 20 g of double distilled water was added, and the pH was determined to be 7.38. The homogenate was hydrolyzed by adding flavourzyme with 4000 U/g protein, at 50° C. for 4 h. Enzyme inactivation (at 100° C. for 10 min) and centrifugation were performed subsequently. After the hydrolysate was successively passed through a common filter paper, 1.2 μm filter membrane, 0.8 μm filter membrane, 2% (volume ratio) activated carbon was added. Then the treated hydrolysate was decolorized at 70° C. for 1 h, centrifuged, filtered through 0.8 μm filter membrane, filtered with celite filter cake, filtered through 0.45 μm filter membrane, ultrafiltered through 10 k ultrafiltration membrane and spray dried to obtain bright white and non-fishy sea cucumber protein peptides.

### Embodiment 2

Cucumaria frondos were eviscerated, washed, air dried and homogenized. 10 g of sea cucumber homogenate was weighed, 30 g of double distilled water was added, and the pH was determined to be 6. The homogenate was hydrolyzed by adding flavourzyme with 6000 U/g protein, at 60° C. for 6 h. Enzyme inactivation (at 95° C. for 15 min) and centrifugation were performed subsequently. After the hydrolysate was successively passed through a common filter paper, 1.2 μm filter membrane, 0.8 μm filter membrane, 0.8% (volume ratio) activated carbon was added. Then the treated hydrolysate was decolorized at 60° C. for 0.5 h, centrifuged under hot, filtered through 0.8 μm filter membrane, filtered with celite filter cake, filtered through 0.45 μm filter membrane, ultrafiltered through 10 k ultrafiltration membrane and spray dried to obtain yellow and slightly fishy sea cucumber protein peptides.

### Embodiment 3

Cucumaria frondos were eviscerated, washed, air dried and homogenized. 10 g of sea cucumber homogenate was weighed, 40 g of double distilled water was added, and the pH was determined to be 8. The homogenate was hydrolyzed by adding flavourzyme with 500 U/g protein, at 40° C. for 0.5 h. Enzyme inactivation (at 98° C. for 12 min) and centrifugation were performed subsequently. After the hydrolysate was successively passed through a common filter paper, 1.2 μm filter membrane, 0.8 μm filter membrane, 3% (volume ratio) activated carbon was added. Then the treated hydrolysate was decolorized at 80° C. for 1.5 h, centrifuged under hot, filtered through 0.8 μm filter membrane, filtered with celite filter cake, filtered through 0.45 μm filter membrane, ultrafiltered through 10 k ultrafiltration membrane and spray dried to obtain bright white and non-fishy sea cucumber protein peptides.

### Embodiment 4

Cucumaria frondos were eviscerated, washed, air dried and homogenized. 10 g of sea cucumber homogenate was weighed, 20 g of double distilled water was added, and the pH was determined to be 7.45. The homogenate was hydrolyzed by adding flavourzyme with 1000 U/g protein, at 50° C. for 1 h. Enzyme inactivation (at 95° C. for 15 min) and centrifugation were performed subsequently. After the hydrolysate was successively passed through a common filter paper, 1.2 μm filter membrane, 0.8 μm filter membrane, 2% (volume ratio) activated carbon was added. Then the treated hydrolysate was decolorized at 75° C. for 1 h, centrifuged under hot, filtered through 0.8 μm filter membrane, filtered with celite filter cake, filtered through 0.45 μm filter membrane, ultrafiltered through 10 k ultrafiltration membrane and spray dried to obtain bright white and non-fishy sea cucumber protein peptides.

### Embodiment 5

Fresh Cucumaria frondos were eviscerated, washed, air dried and homogenized. 10 g of sea cucumber homogenate was weighed, 30 g of double distilled water was added, and the pH was determined to be 7.36. The homogenate was hydrolyzed by adding flavourzyme with 2000 U/g protein, at 50° C. for 4 h. Enzyme inactivation (at 95° C. for 15 min) and centrifugation were performed subsequently. After the hydrolysate was successively passed through a common filter paper, 1.2 μm filter membrane, 0.8 μm filter membrane, 2% (volume ratio) activated carbon was added. Then the treated hydrolysate was decolorized at 75° C. for 1 h, centrifuged under hot, filtered through 0.8 μm filter membrane, filtered with celite filter cake, filtered through 0.45 μm filter membrane, ultrafiltered through 10 k ultrafiltration membrane and spray dried to obtain bright white and non-fishy sea cucumber protein peptides.

### Embodiment 6

Fresh Cucumaria frondos were eviscerated, washed, air dried and homogenized. 10 g of sea cucumber homogenate was weighed, 20 g of double distilled water was added, and the pH was determined to be 7.38. The homogenate was hydrolyzed by adding flavourzyme with 1000 U/g protein, at 50° C. for 0.5 h. Enzyme inactivation (at 97° C. for 13 min) and centrifugation were performed subsequently. After the hydrolysate was successively passed through a common filter paper, 1.2 μm filter membrane, 0.8 μm filter membrane, 1.5% (volume ratio) activated carbon was added. Then the treated hydrolysate was decolorized at 70° C. for 1 h, centrifuged under hot, filtered through 0.8 μm filter membrane, filtered with celite filter cake, filtered through 0.45 μm filter membrane, ultrafiltered through 10 k ultrafiltration membrane and spray dried to obtain light white and non-fishy sea cucumber protein peptides.

### Embodiment 7

Fresh Cucumaria frondos were eviscerated, washed, air dried and homogenized. 20 g of sea cucumber homogenate was weighed, 40 g of double distilled water was added, and the pH was determined to be 7.56. The homogenate was hydrolyzed by adding flavourzyme with 4000 U/g protein, at 48° C. for 5 h. Enzyme inactivation (at 100° C. for 15 min) and centrifugation were performed subsequently. After the hydrolysate was successively passed through a common filter paper, 1.2 μm filter membrane, 0.8 μm filter membrane, 1.5% (volume ratio) activated carbon was added. Then the treated hydrolysate was decolorized at 65° C. for 1.5 h, centrifuged under hot, filtered through 0.8 μm filter membrane, filtered with celite filter cake, filtered through 0.45 μm filter membrane, ultrafiltered through 10 k ultrafiltration membrane and spray dried to obtain light white and non-fishy sea cucumber protein peptides.

### Embodiment 8

Fresh Cucumaria frondos were eviscerated, washed, air dried and homogenized. 10 g of sea cucumber homogenate was weighed, 20 g of double distilled water was added, and the pH was determined to be 7.13. The homogenate was hydrolyzed by adding flavourzyme with 4000 U/g protein, at 45° C. for 4 h. Enzyme inactivation (at 100° C. for 15 min) and centrifugation were performed subsequently. After the hydrolysate was successively passed through a common filter paper, 1.2 μm filter membrane, 0.8 μm filter membrane, 2% (volume ratio) activated carbon was added. Then the treated hydrolysate was decolorized at 80° C. for 1.5 h, centrifuged under hot, filtered through 0.8 μm filter membrane, filtered with celite filter cake, filtered through 0.45 μm filter membrane, ultrafiltered through 10 k ultrafiltration membrane and spray dried to obtain bright white and non-fishy sea cucumber protein peptides.

### Embodiment 9

Fresh Cucumaria frondos were eviscerated, washed, air dried and homogenized. 20 g of sea cucumber homogenate was weighed, 50 g of double distilled water was added, and the pH was determined to be 7.98. The homogenate was hydrolyzed by adding flavourzyme with 6000 U/g protein, at 60° C. for 6 h. Enzyme inactivation (at 100° C. for 15 min) and centrifugation were performed subsequently. After the hydrolysate was successively passed through a common filter paper, 1.2 μm filter membrane, 0.8 μm filter membrane, 0.5% (volume ratio) activated carbon was added. Then the treated hydrolysate was decolorized at 80° C. for 0.5 h, centrifuged under hot, filtered through 0.8 μm filter membrane, filtered with celite filter cake, filtered through 0.45 μm filter membrane, ultrafiltered through 10 k ultrafiltration membrane and spray dried to obtain yellow and slightly fishy sea cucumber protein peptides.

In order to further verify that the sea cucumber protein peptides prepared by the present disclosure has an excellent technical effect, the hydrolysis degree, molecular weight and the antioxidant evaluation test of the hydrolysate were also conducted on the sea cucumber protein peptides prepared in Embodiments 1˜9. The specific measurement results are shown in Table 4 below.

TABLE 4
Hydrolysis degree, molecular weight and antioxidant evaluation data of the
hydrolysate of the sea cucumber protein peptides prepared in Embodiments 1~9

| | hydrolysis | molecular | O²⁻• | •OH | |
|---|---|---|---|---|---|
| | degree | weight/Mw | clearance | clearance | |
| No. | (%) | (Da) | (%) | (%) | Product quality |
| Embodiment 1 | 42.53 | 657 | 18.34 | 23.01 | Bright white, non-fishy |
| | | | | | and clear after |
| | | | | | dissolving in water |
| Embodiment 2 | 37.87 | 365 | 15.24 | 23.67 | Yellow, slightly fishy |
| | | | | | and clear after |
| | | | | | dissolving in water |
| Embodiment 3 | 24.04 | 3366 | 6.11 | 13.85 | Bright white, non-fishy |
| | | | | | and clear after |
| | | | | | dissolving in water |
| Embodiment 4 | 5.47 | 2680 | 7.24 | 14.12 | Bright white, non-fishy |
| | | | | | and clear after |
| | | | | | dissolving in water |
| Embodiment 5 | 5.47 | 562 | 13.54 | 21.72 | Bright white, non-fishy |
| | | | | | and clear after |
| | | | | | dissolving in water |
| Embodiment 6 | 5.79 | 3345 | 5.87 | 13.56 | Bright white, non-fishy |
| | | | | | and clear after |
| | | | | | dissolving in water |
| Embodiment 7 | 24.03 | 690 | 14.11 | 20.32 | Light white, non-fishy |
| | | | | | and clear after |
| | | | | | dissolving in water |
| Embodiment 8 | 19.16 | 6753 | 5.53 | 13.35 | Bright white, non-fishy |
| | | | | | and clear after |
| | | | | | dissolving in water |
| Embodiment 9 | 14.91 | 5380 | 5.12 | 12.47 | Yellow, slightly fishy |
| | | | | | and clear after |
| | | | | | dissolving in water |

It is known from Table 4 above that sea cucumber protein peptides prepared by the method disclosed were basically non-fishy, and from the results, it can be seen that the smaller the molecular weight, the higher the ability of oligopeptides (400-500 Da) to scavenge hydroxyl radicals. And the molecular weight distribution of sea cucumber peptides was closely related to their antioxidant activities, and the ability of sea cucumber protein peptides to scavenge superoxide anions improved with the decrease of molecular weight.

The above description of the disclosed embodiments enables the skilled in the art to achieve or use the disclosure. Multiple modifications to these embodiments will be apparent to those skilled in the art, and the general principles defined herein may be achieved in other embodiments without departing from the spirit or scope of the disclosure. The present disclosure will therefore not be restricted to these embodiments shown herein, but rather to comply with the broadest scope consistent with the principles and novel features disclosed herein.

We claim:

1. A preparation method of sea cucumber protein peptides, comprising:
(1) pretreatment of raw material: eviscerating, washing, drying and grinding fresh Cucumaria frondos into a homogenate;
(2) enzymolysis: adding water to the homogenate of step (1) and determining pH, and then adding a protease for enzymolysis to obtain a sea cucumber peptide hydrolysate;
(3) purification and drying: subjecting the sea cucumber peptide hydrolysate of step (2) to boiling water bath to inactivate enzyme, followed by filtering, decolorizing and spray drying to obtain sea cucumber protein peptides.

2. The preparation method of claim 1, wherein the protease in step (2) is a flavourzyme, a papain, a pancreatin, a neutral protease or an alkaline protease, and an addition of the protease is 500˜6000 U/g.

3. The preparation method of claim 1 or 2, wherein in step (2), an enzymolysis temperature is 40° C.˜60° C., and an enzymolysis time is 0.5˜6 h.

4. The preparation method of claim 3, wherein a mass ratio of the homogenate to the water is 1:(2˜2.5), and the pH of the enzymolysis is 6˜8.

5. The preparation method of claim 1, wherein in step (3), a temperature of the boiling water bath is 95° C.˜100° C., and a time of enzyme inactivation is 10˜15 min.

6. The preparation method of claim 1, wherein a specific operation of the purification is as follows: passing the hydrolysate through an ordinary filter paper, an 1.2 μm filter membrane and an 0.8 μm filtering membrane in turn, adding a decolourant into the filtered hydrolysate for decolorization, centrifuging the decolorized hydrolysate under hot, passing the centrifuged hydrolysate through an 0.8 μm filtering membrane, and then filtering with an celite filter cake, after passing through a 0.45 μm filter membrane, ultrafiltering with a 10 k ultrafiltration membrane to obtain a purification filtrate.

7. The preparation method of claim 6, wherein the decolourant is activated carbon or diatomite, and an addition of the decolourant is 1˜3% of the volume of the hydrolysate, and a decolorizing temperature is 60° C.˜80° C., and a decolorizing time is 0.5˜1.5 h.

8. An application of the sea cucumber protein peptides prepared by the method of claim 1 in food and daily cosmetics.
